(19) United States
(12) Patent Application Publication
(10) Pub. No.: US 2013/0238065 A1
(43) Pub. Date: Sep. 12, 2013

(54) EPIDERMAL COOLING

(71) Applicant: Arvind M. Rao, Rochester, NY (US)

(72) Inventor: Arvind M. Rao, Rochester, NY (US)

(73) Assignee: EXERT CO., Wilmington, DE (US)

(21) Appl. No.: 13/412,919

(22) Filed: Mar. 6, 2012

(51) Int. Cl. A61F 7/00; A61K 33/00; A61K 33/44; A61P 17/00; A61K 33/24; A61K 33/38; A61K 33/08; B05D 7/00; A61K 33/12

(52) U.S. Cl. 607/112; 427/2.31; 424/722; 424/125; 424/724; 424/683; 424/617; 424/618; 424/691; 424/692; 424/653

(57) ABSTRACT

A phase change formulation includes a reversible phase change material and a thermal conductivity additive formulated for application to a user's epidermis.

### BACKGROUND OF THE INVENTION

1. Field of the Invention

This invention relates to cooling. More specifically, the invention relates to a formulation including a phase change material provided for intimate contact with a user's epidermis to cool the epidermis in response to increased heat.

2. Description of Related Art

Several products are commercially available that are intended to combat uncomfortable sensations experienced in response to bodily heating. For example, patches containing menthol or some other similar substance are provided for application by a user to the user's epidermis, to impart on the user a cooling sensation. Such patches are marketed for application to the forehead when running a fever or to an injury site, such as a sprain. Other sources of increased epidermal temperature also are known, for example, due to exercise, drug side effects or hormonal imbalances, including as a result of menopause.

Menthol-based and similar conventional cooling solutions, however, are single use. Menthol creates the illusion of cooling because it activates TRPM8, a protein in the body that perceives cold. Once the menthol dissipates, it is gone though. Moreover, because menthol is only “tricking” the body into feeling cool, it does not actually create any epidermal temperature change.

Thus, there is a need for reusable or reversible cooling relief that a user can apply to a desired site on the epidermis.

There is a further need for fast-acting cooling relief with continued efficacy over an extended period of time.

There also is a need for cooling relief that lowers the temperature of the epidermis.

### BRIEF SUMMARY OF THE INVENTION

This invention addresses these needs by providing improvements in epidermal cooling.

In one aspect of the invention, a wearable phase change composition is provided to provide cooling to the wearer. The composition includes a reversible phase change material and a thermal conductivity additive.

In another aspect of the invention, a cooling device includes a substrate having an adhesive on a first side and a phase change composition on a second side of the adhesive, opposite the first side. The phase change composition includes a phase change material and a thermal conductivity additive.

In yet another aspect of the invention, a method of making a cooling device includes providing a predetermined amount of a phase change composition on a substrate and drying the phase change composition to remove substantially all of the water from the composition.

An understanding of these and other aspects, features, and benefits of the invention may be had with reference to the following disclosure, in which preferred embodiments of the invention are described.

### BRIEF DESCRIPTION OF THE DRAWING FIGURES

FIG. 1 is a schematic illustration of a phase change composition according to one embodiment of the invention.

FIGS. 2A and 2B are, respectively, isometric and cross-sectional views of a cooling device according to one embodiment of the invention.

FIG. 3 is flow chart illustrating a process for making a cooling device.

FIG. 4 is a schematic illustration showing a location on the body on which embodiments of this invention were tested.

FIG. 5 is a box plot of field test results achieved using a cooling device according to an embodiment of the invention.

### DETAILED DESCRIPTION OF THE INVENTION

As noted above, the invention generally relates to epidermal cooling and more specifically to wearable cooling compositions that include reversible phase change materials, rendering them reusable.

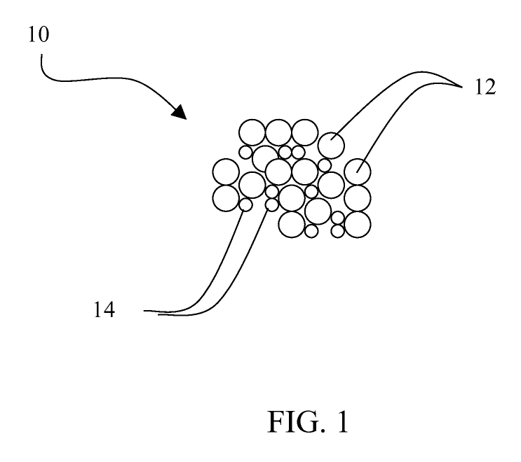

In one embodiment, the invention includes a phase change composition 10 illustrated schematically in FIG. 1. The formulation 10 includes a phase change material 12 and a thermal conductivity agent 14.

The phase change material is preferably a reversible phase change material. That is, it can readily change back-and-forth between two states with applied temperatures. A conventional construction of such phase change materials includes a core material encapsulated in an outer shell. The core material is chosen for its specific melting point, which usually ranges from about −30-degrees Celsius to about 55-degrees Celsius. The core material is preferably solid at ambient temperature. It may be an n-alkane. Such n-alkanes include heptadecane (which has the formula C₁₇H₃₆ and a melting point of about 22-degrees Celsius), octadecane (C₁₈H₃₈; 28° C.), nonadecane (C₁₉H₄₀; 32° C.), eicosane/icsosane (C₂₀H₄₂; 36.8° C.), and heneicosane (C₂₁H₄₄; 40.5° C.). The outer shell is usually made of an inert, stable polymer or plastic. Preferred phase change materials are generally spherical in shape and have a mean particle size on the order of about 1 to about 50 microns, with about 10 to about 20 microns being more preferred. Phase change materials such as those just described are commercially available, including from MicroTek Laboratories, Inc. of Dayton, Ohio and BASF Corporation of Ludwigshafen, Germany. Encapsulated phase change materials generally are available in dry and wet formulations, with the wet formulations containing the phase change material and added water usually at about 30%.

The thermal conductivity agent may be any of a number of materials, and preferably has a thermal conductivity greater than about 7 W/mK. The inventor has experimented with additives including beryllium oxide (having a thermal conductivity of about 272 W/mK), quartz (12 W/mK), natural diamond (2200 W/mK), talc (10.7 W/mK), titanium dioxide (8.4 W/mK), magnesium oxide (30-48 W/mK), graphene (4500-5300 W/mK), carbon nanotubes (3100-3500 W/mK), silver (400 W/mK), aluminum oxide (25-36 W/mK), and bismuth (7.9 W/mK). Other thermal conductivity additives also may be used without departing from the spirit and scope of the invention.

The inventor has found that the combination of the phase change material with the thermal conductivity agent leads to a greater cooling effect for the wearer, and more quickly. Whereas the encapsulated phase change material will take time to melt upon application to a wearer's skin, the thermal conductivity agent immediately begins to conduct heat away from the user's skin. This conducted heat will immediately begin to cool the user's skin. Moreover, because the thermal conductivity additive is dispersed among the phase change material, the phase change material is more readily heated up, resulting in a quicker melting of the core material of the phase change material. As the core melts, the accompanying endothermic reaction absorbs heat, creating a temperature decrease around the phase change material, further cooling the user's skin situated proximate the composition. The thermal conductivity agent further conducts this cooling to the skin.

To achieve the above-described benefits, the composition 10 of FIG. 1 is intended to be placed in close proximity to, or in intimate contact with, the user's epidermis. That is, the composition 10 is intended to be “worn” by the user. The composition 10 may be contained in a gel, cream, paste, lotion, or powder, or provided as a peel, mask, tape or patch.

Although not illustrated in FIG. 1, and depending upon the application, additives may be included in the phase change composition. For example, in the case of a gel, cream, paste or lotion, additives such as pharmaceutical grade waxes, glycerin and petroleum jelly may be used included to contain the phase change composition. Other substances, such as polyethylene glycol, propylene glycol, polyoxyethylene derivatives of sorbitan monolaurate, available under the tradename Tween, cyclopentasiloxane, cyclomethicone, dimethicone and cyclomethicone-dimethicone crosspolymer blends also may be included to emulsify or promote cohesion of the constituent parts of the composition. Again, for example, in the case of a powder, substances such as rice powder, aluminum sesquichlorohydrate, mineral oil, cyclomethicone-dimethicone crosspolymer blends may be included to enhance the end user experience. Again, for example in the case of a peel, mask, tape or patch, a binder or thickener may be included to provide a desired viscosity to the composition. Binders or thickeners, such as but not limited to polyacrylic acid (PAA), polyvinylpyrrolidone (PVP), carboxymethyl cellulose may be used. Other substances such as polyvinyl alcohol, polyethylene glycol, propylene glycol and polyoxyethylene derivatives of sorbitan monolaurate, available under the tradename Tween, also may be included to emulsify or promote cohesion of the constituent parts of the composition. Other additives such as diluents (for example distilled or deionized water), coloring agents, humectants (for example glycerin), antioxidants, rheology stabilizers, chelating agents, preservatives and fragrances or the like also may be included. Furthermore, these additives are often available commercially as facial peels or masks in a pre-blended or concentrated form. Such concentrates afford the expedient blending of the inventive phase change composition ingredients, the phase change material and thermal conductivity agent, to create the final compositions. Concentrates such as those just described are commercially available, including from pH Beauty Labs, Inc. of Los Angeles, Calif., hereby called “Peel concentrate” containing water, polyvinyl alcohol, isopropyl alcohol, glycerin, polyethylene glycol, propylene plycol, polysorbate 20, polyvinylpyrrolidone, panthenol, allantoin, aloe barbadensis leaf juice, cucumber extract, shea butter, disodium EDTA, ethyl lactate, ascorbic acid, thiotaine, tocopheryl acetate, butylene glycol, hydrolyzed silk, methylparaben, propylparaben, Blue 1, citronellol, hexyl cinnamal, butylphenyl methylpropional.

Compositions made in accordance with this disclosure generally include up to about 40% encapsulated phase change materials, with the balance being thermal conductivity additive(s) or thermal conductivity additive(s) and additional additives. More preferably, the phase change material is present in the composition in an amount from about 15% to about 50%.

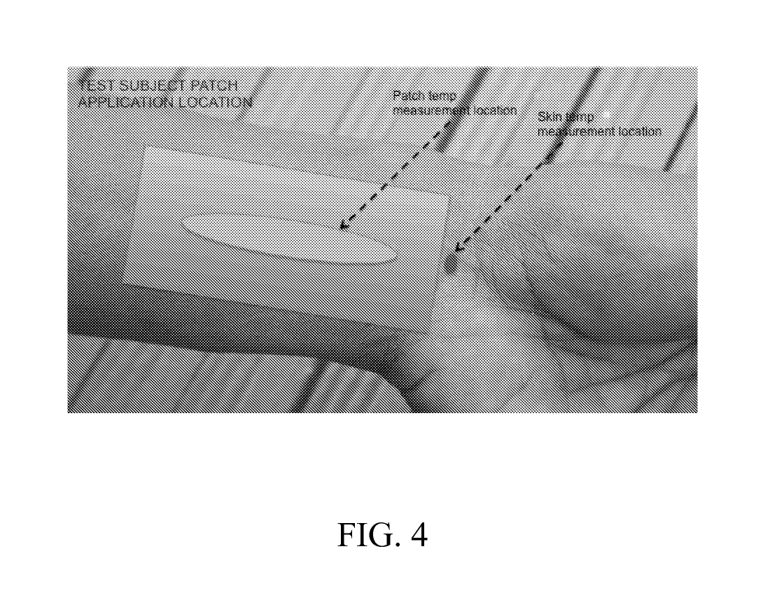

The inventor has experimented with a number of delivery modalities for phase change compositions like those described above. As will be appreciated, some of the applications were wet application, e.g., lotions, peels, or gels, to which water was added, and others were dry, e.g., creams and adhesive patches or tapes. In each example, the product was applied covering an equivalent amount of surface area on the wearer's inner arm, between the wrist and elbow as detailed in FIG. 4.

EXAMPLE 1 provides epidermal cooling using the composition 10 of FIG. 1 in the form of a gel commonly administered to the skin by a deodorant stick or roll-on. 28.2 g of MCPM-37D (a microencapsulated phase change material based on eicosane with a melting point of 37° C. commercially available from MicroTek Laboratories, Inc.) and 17.5 g of cyclopentasiloxane-dimethiconol blend (DC 1501 Fluid from Dow Corning of Midland, Mich.) were combined and stirred at approximately 60 RPM for 30 seconds followed by 80 RPM for 60 seconds. To this 12.2 g of distilled water and 23.7 g of propylene glycol were added and stirred at 80 RPM for 2 minutes. To this 18.4 g of talc was added and stirred at 80 RPM for 5 minutes to form a light fluffy opaque white gel.

Comparative EXAMPLE C1 is similar to Example 1 but with the exclusion of the thermal conductivity agent. In this way the net benefit of the thermal conductivity agent in concert with the phase change materials may be elucidated. 32.2 g of MCPM-37D (microencapsulated phase change material based on eicosane with a melting point of 37° C.) and 19.0 g of cyclopentasiloxane-dimethiconol blend (DC 1501 Fluid) were combined and stirred at approximately 60 RPM for 30 seconds followed by 80 RPM for 60 seconds. To this 25.5 g of distilled water and 23.3 g of propylene glycol were added and stirred at 80 RPM for 5 minutes to form a light fluffy opaque white gel.

EXAMPLE 2 provides epidermal cooling in the form of a powder commonly administered to the skin by hand or cosmetic makeup brush. 40 g of MPCM-FF28D (formaldehyde-free microencapsulated phase change material based on octadecane with a melting point of 28° C. commercially available from MicroTek Laboratories, Inc.), 48 g of talc, 10 g of aluminum sesquichlorohydrate powder (Reach 301 from Summit Reheis Corporation of Huguenot, N.Y.) and 2 g of cyclomethicone-dimethicone crosspolymer blend (DC 9045 Silicone Elastomer Blend from Dow Corning) were combined and stirred at approximately 60 RPM for 4 minutes resulting in a white, free flowing and no-dusting powder.

Comparative EXAMPLE C2 contains commercially available talcum powder sold by Johnson & Johnson under the Johnson's Baby Powder tradename.

EXAMPLE 3 provides epidermal cooling in the form of an adhesive patch or tape. 4.8 g of talc, 38.1 g of MPCM-28D (microencapsulated phase change material based on octadecane with a melting point of 28° C. commercially available from MicroTek Laboratories, Inc.) and 9.5 g of propylene glycol were combined and stirred at approximately 60 RPM for 30 seconds. 38.1 g of peel concentrate (pH Beauty Labs, Inc. of Los Angeles, Calif.) and 9.5 g of distilled water were then added to the above ingredients and stirred at 80 RPM for 60 seconds followed by 5 minutes at 120 RPM forming in a thick, high viscosity paste.

EXAMPLE 4 provides epidermal cooling in the form of an adhesive patch or tape. 4.8 g of magnesium oxide, 38.1 g of MPCM-28D (microencapsulated phase change material based on octadecane with a melting point of 28° C.) and 9.5 g of propylene glycol were combined and stirred at approximately 60 RPM for 30 seconds. 38.1 g of peel concentrate and 9.5 g of distilled water were then added to the above ingredients and stirred at 80 RPM for 60 seconds followed by 5 minutes at 120 RPM forming in a thick, high viscosity paste.

Comparative EXAMPLE C3 contains 100 g of peel concentrate only. It has no phase change material or thermal conductivity additive.

Comparative EXAMPLE C4 includes a phase change material, but excludes the thermal conductivity additive. Specifically, the formulation includes 21 g of MPCM-37D (microencapsulated phase change material based on eicosane with a melting point of 37° C.), 70 g of peel concentrate and 9 g of distilled water were combined and stirred at approximately 60 RPM for 30 seconds followed by 5 minutes at 80 RPM forming in a thick, high viscosity paste.

The patches of Examples 3, 4, C3 and C4 were embodied in the same way, i.e., composition on substrate, but as will be appreciated from Table 1, exhibited very different results. The formulation with both phase change material and thermal conductivity additive were very effective at cooling, while the compositions without either or with only the phase change material had little to no cooling effect.

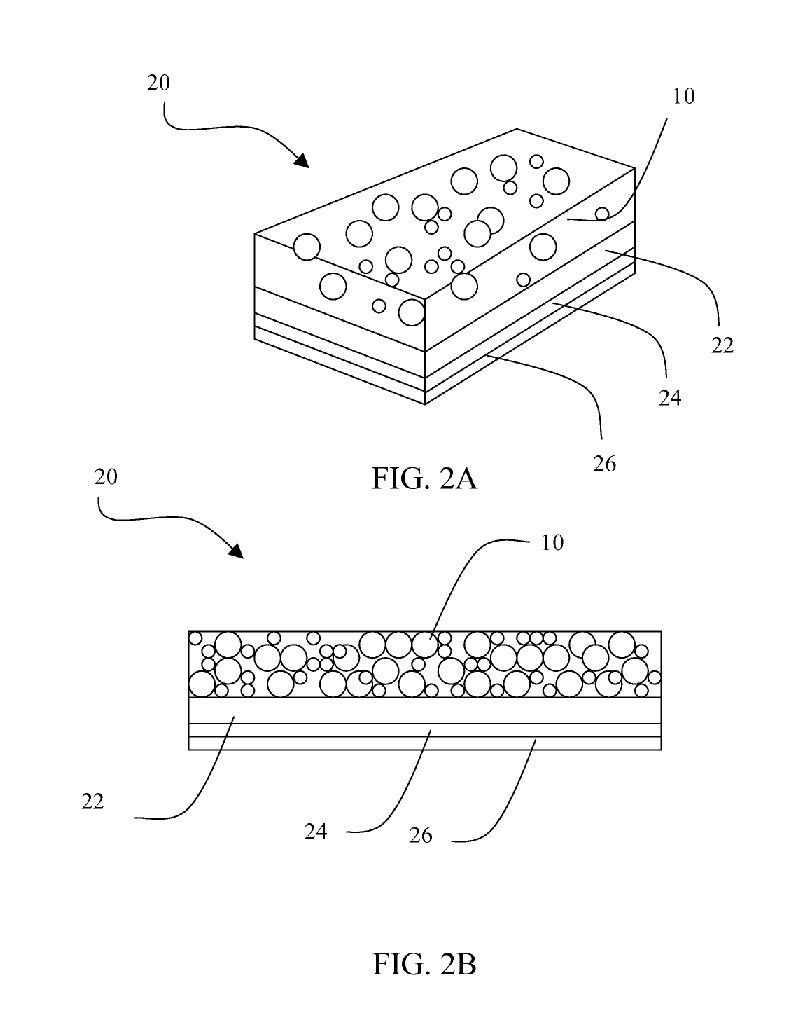

FIGS. 2A and 2B illustrate an example of the adhesive tape prepared from the composition in Examples 3 and 4. In those figures, a phase change composition 10, which may be the same phase change composition as that illustrated in FIG. 1, is a wearable patch 20, which is part of a longer tape. The formulation 10 is disposed on a substrate 22. Adhesive 24 is provided on the substrate 22, on a side opposite the formulation 10. This adhesive 24 is provided to adhere the patch 20 to a user's epidermis. A removable backing layer 26 also is provided, which is removed by the user to expose the adhesive 24, as preparation for application to the skin.

The applications were as illustrated in FIGS. 2A and 2B, with the substrate 22, adhesive 24, and backing layer 26 being provided by a commercially available adhesive transfer tape PS-1243, sold by Polymer Science Inc., of Monticello, Ind. The formulation was deposited on the transfer tape.

Such compositions were applied to the skin, with the wearer being asked to rate the perceived temperature of their skin just prior to application (initial rating), upon application (0 minutes) and at subsequent five minute time intervals for up to 30 minutes, on the following Likert bipolar scale: 1—extremely warm, 2—moderately warm, 3—slightly warm, 4—no feeling of warm or cold, 5—slightly cool, 6—moderately cool, 7—extremely cool, may feel some tingling. As will be appreciated by reference to Table 1, of the all the application forms, the adhesive tape applications were particularly effective at lowering the wearer's skin temperature. Further note the increased rate at which the cooling sensation develops and its duration as a function of the combination of the phase change materials and thermal conductivity agent. When specifically applied as a patch, the cooling performance of these compositions is further enhanced by the thermal conductivity of the thermal conductivity agent as evidenced when Example 4 is compared with Example 3. This increased thermal conductivity is further elucidated by laboratory thermal conductivity measurement of patches at 30-32° C.

TABLE 1

| | Examples - Cooling Sensation Ratings | | | | | | | |
|---|---|---|---|---|---|---|---|---|
| | 1 | C1 | 2 | C2 | 3 | 4 | C3 | C4 |
| PCM | Yes | Yes | Yes | No | Yes | Yes | No | Yes |
| TC Additive | Yes | No | Yes | Yes | Yes | Yes | No | No |
| Product | Gel - | Gel - | Powder - | Powder - | Adhesive | Adhesive | Adhesive | Adhesive |
| form | wet | wet | dry | dry | patch - | patch - | patch - | patch - |
| | | | | | dry | dry | dry | dry |
| Before | 3 | 3 | 3 | 3 | 3 | 3 | 3 | 3 |
| Application | | | | | | | | |
| (B4) | | | | | | | | |
| Upon | 4 | 4 | 5 | 5 | 5 | 5 | 4 | 4 |
| Application | | | | | | | | |
| 0 min | | | | | | | | |
| 5 min | 4 | 4 | 5 | 4 | 4 | 6 | 4 | 4 |
| 10 min | 4 | 4 | 5 | 4 | 4 | 6 | 4 | 4 |
| 15 min | 5 | 4 | 5 | 4 | 5 | 6 | 3 | 4 |
| 20 min | 5 | 4 | 5 | 3 | 6 | 6 | 3 | 4 |
| 25 min | 5 | 4 | 5 | 3 | 6 | 7 | 3 | 4 |
| 30 min | 5 | 4 | 5 | 3 | 6 | 7 | 3 | 3 |
| Rating | 2 | 1 | 2 | 0 | 3 | 4 | 0 | 0 |
| difference | | | | | | | | |
| [30 min − B4] | | | | | | | | |
| Thermal | — | — | — | — | 0.171 | 0.184 | — | 0.097 |
| Conductivity | | | | | | | | |
| of patch | | | | | | | | |
| [W/mK] | | | | | | | | |

Thermal Conductivity Measurement by ASTM E1530-11 at 30-32° C.

Encouraged by the surprising initial results of the adhesive tape, or patch, formulations on a small sample size, the inventor conducted further tests of the adhesive tape prepared from Example 3.

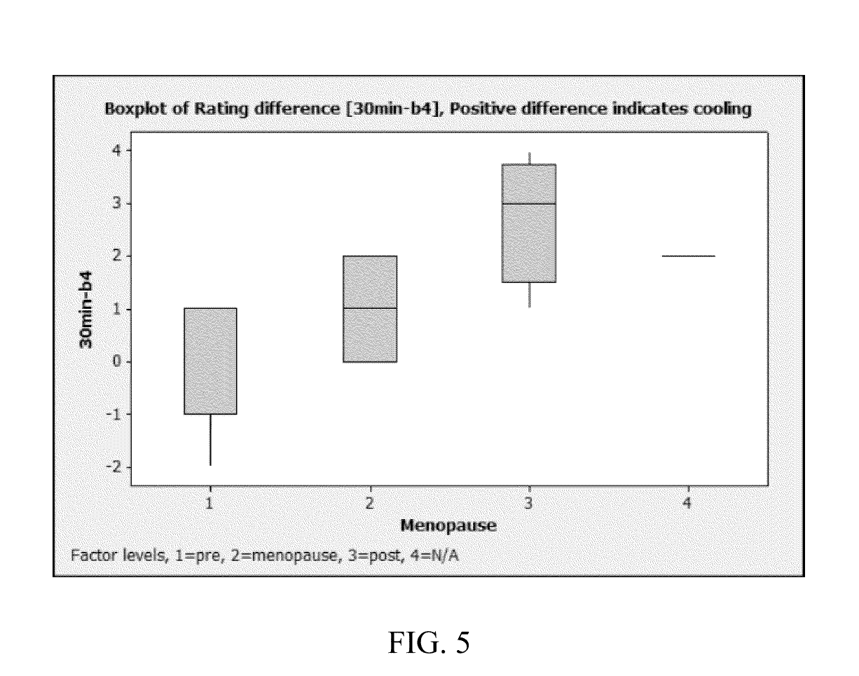

The so-formed patches (based on Example 3) were then field tested on women between the ages of 40 and 60, all of whom were experiencing hot flashes. Of them, some were pre-menopausal, some were menopausal, and some were post-menopausal, as each of those terms is clinically defined. Still others experienced hot flashes because of medication side-effects or some other cause. As with the test described above, each participant in the trial was asked to describe their skin temperature on a scale of 1 to 7 as follows: 1—extremely warm, 2—moderately warm, 3—slightly warm, 4—no feeling of warm or cold, 5—slightly cool, 6—moderately cool, and 7—extremely cool, may feel some tingling. This data was recorded just before application of the patch to the wearer, upon application of the patch and then at five-minute intervals up to and including 30 minutes. The use of a standardized rating Likert-type scale allows for the comparison of cooling sensation from the beginning of the test to any time segment thereafter. After all data was collected, a paired t-test of the group's mean cooling sensation rating after 30 min [30 min Rating] compared to the group's initial rating [b4 Rating] shows that the cooling effect of the patch as described on FIGS. 2A and 2B is statistically significant with a p<0.01 (n=12). The group's 30 min Rating mean=5.18 and the group's initial rating [b4 Rating]=3.54 resulting in a mean difference of +1.64 units. Note that this statistical result was obtained with women of different subgroups (e.g. premenopausal, menopausal, post-menopausal and other). The data was further analyzed via a one-way analysis of variance (ANOVA) test with the subgroup category (e.g. premenopausal, menopausal, post-menopausal and other) as the primary source of variation in the cooling sensation difference [30 min Rating−b4 Rating] (F-stat=2.97, p=0.097 and r²=52.7%). A box-plot of the data is illustrated in FIG. 5.

It is clear from the graph that post-menopausal women experienced the greatest cooling effect from the patch. A single, pre-menopausal woman tested experienced warming from the patch.

In addition to asking the wearer her perceived cooling, the inventor also measured the wearer's skin temperature just prior to application of the patch and upon removal of the patch at 30 minutes from application. A mean temperature decrease of 1.8° F. was measured across all wearers significant to p<0.01. The temperature of the surface of the patch contacting the skin was also measured before application and upon removal at 30 minutes from application. A mean temperature increase of 5.8° F. was measured across all patches significant to p<0.01.

The findings of this study are particularly encouraging in that they are statistically significant. The wearers experienced cooling, easing the discomfort associated with their hot flashes. The combination of the thermal conductivity additive and the reversible phase change material provided immediate cooling to a user, and continued to cool for at least 30 minutes in most applications. Moreover, after removing the patch and placing in a cool environment, such as a refrigerator or freezer, the phase change material re-solidifies, ready for another use.

The inventor also believes that other formulations may work better for different users. The pre-application temperature of women who are post-menopausal was observed to be lower than women who are either pre-menopausal or menopausal. Other combinations of reversible phase change materials and thermal conductivity additives may be better suited for these other women. For example, a phase change material with a higher melting point, more of the thermal conductivity additive, or a more conductive thermal conductivity additive may achieve better results for pre-menopausal or menopausal women. Moreover, a formulation could include two or more phase change materials, each having a different melting point, which could allow a single product to work for a number of wearer's. More than one phase change material would also provide cooling over a greater range of temperatures, for example, if the wearer's skin gets significantly warmer. Other modifications also will be appreciated by those of ordinary skill in the art.

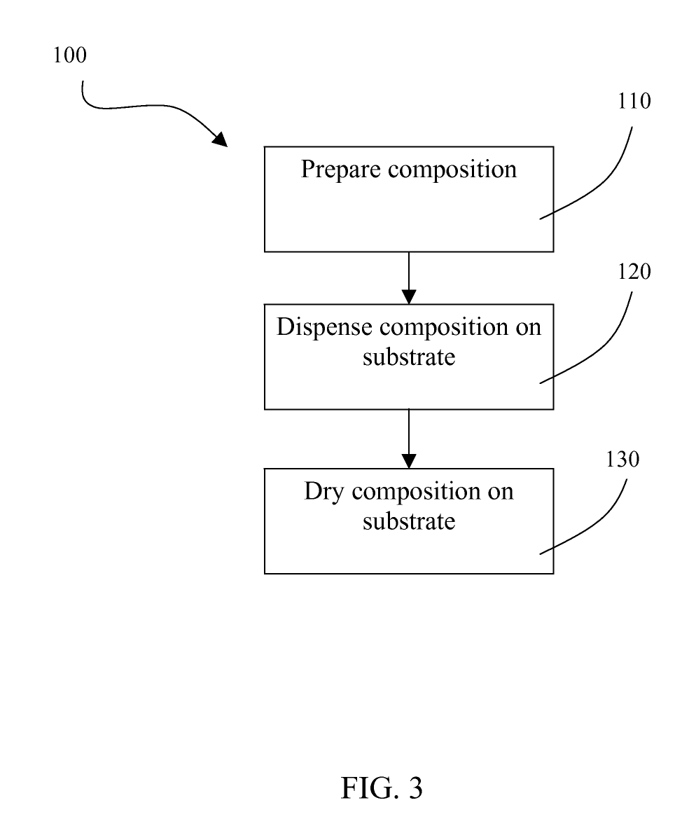

A method of making patches such as those illustrated in FIGS. 2A and 2B now will be described with reference to the flow chart of FIG. 3. There, a method 100 includes a first step 110 of preparing the phase change composition. In the simplest case, the composition is prepared by mixing the reversible phase change material and the thermally conductive additive together with any other additives. The composition made for the field testing described above included as additives, a binder, propylene glycol, and water.

In step 120, the composition is dispensed onto a substrate. As noted above with respect to the patches made for field testing, an adhesive transfer tape, commercially available from Polymer Science Inc., of Monticello, Ind., for example, may be used as the substrate. Commercially available tape has the advantage of being approved and accepted for use in wearable applications. Of course, any substrate could be used, but a preferred substrate will not significantly insulate the wearer from cooling benefits of the formulation. Conversely, adhesive transfer tapes that have enhanced thermal conductivity and commercially available from 3M Corporation, Polymer Sciences Inc., and others are of further advantage as they enhance the thermal conductivity of the entire wearable application. The composition is generally present on the substrate in a thickness of between about 0.5 mm and about 10 mm, and more preferably between about 1 mm and about 1.5 mm.

In step 130, the composition-on-substrate is dried. Preferably, drying takes place at an elevated temperature, such as in an oven. The drying process preferably drives off substantially all water remaining in the composition. In preferred embodiments, the water content will be less than about 10% of the formulation, and more preferably less than about 5%. Water may act to provide non-reversible cooling, as it may also evaporate upon application to the skin. As such, compositions including a large amount of water likely will operate much better the first time than they will in subsequent uses. By substantially eliminating water, the composition will perform nearly identically for each application.

After drying, the patch is ready for use. The method may include other steps, too. For example, the surface of the composition may be textured to effectively increase the surface area of the composition. Moreover, a barrier layer may be placed over the composition, such that the composition is bounded both on top (by the barrier layer) and the bottom (by the substrate).

In yet another embodiment, the patch may not include adhesive at all. For example, the patch could be affixed to the user's skin with a wrap, brace, or the like. As noted above, compositions and embodiments according to the invention are reusable because the phase change is encapsulated. Adhesive may lose its tackiness over multiple uses, so embodying the invention in a wrap, for example, will allow the user the benefit of repeated uses.

The invention has been described thus far as having a cooling effect on the wearer. This is not necessary. A phase change composition according to the invention could alternatively be formulated that is intended to warm the user. For example, a wearable composition formulated according to the above-described embodiments and methods may include a reversible phase change material having a melting point at or below ambient temperature. Whenever the temperature dropped below the melting point, the exothermic reaction accompanying the phase change from liquid to solid would give off heat, warming the wearer. Outdoor, cold-weather applications are an example of a contemplated use of such a warming composition.

While the invention has been described in connection with several presently preferred embodiments thereof, those skilled in the art will appreciate that many modifications and changes may be made therein without departing from the true spirit and scope of the invention which accordingly is intended to be defined solely by the appended claims.

1) A wearable phase change composition comprising:
a reversible phase change material and a thermal conductivity additive formulated for application to a user's epidermis.

2) The composition of claim 1, wherein the thermal conductivity additive is chosen from a list comprising beryllium oxide, quartz, natural diamond, talc, titanium dioxide, graphene, carbon nanotubes, silver, aluminum oxide, magnesium oxide, and bismuth.

3) The composition of claim 1, wherein the phase change material is an encapsulated phase change material.

4) The composition of claim 1, wherein the phase change material is an encapsulated reversible phase change material.

5) The composition of claim 4, wherein the phase change material changes phase at between about 20-degrees Celsius and about 41-degrees Celsius.

6) The composition of claim 4, wherein the material changes phase from solid to liquid.

7) The composition of claim 1 provided as one of a powder, an adhesive-backed tape, a cream, a peel, a gel, and a lotion

8) The composition of claim 1, wherein the composition is dried to remove substantially all water therefrom.

9) The composition of claim 1, wherein the composition is dried to remove less than about 5% of all water therefrom.

10) A cooling device comprising:
a substrate having an adhesive on a first side;
a phase change composition provided on a second side of the adhesive, opposite the first side, the phase change composition including a reversible phase change material.

11) The cooling device of claim 10, wherein the phase change composition further comprises a thermal conductivity additive.

12) The cooling device of claim 10, further comprising a film disposed on the phase change composition, on a side of the phase change composition opposite the substrate.

13) The cooling device of claim 10, further comprising a removable backing covering the adhesive.

14) The cooling device of claim 10, wherein a surface of the phase change composition opposite the substrate is textured.

15) The cooling device of claim 10, wherein the phase change composition disposed on the substrate has a thickness between about 0.5 and about 10 mm.

16) The cooling device of claim 15, wherein the thickness is between about 1 and about 1.5 mm.

17) A method of making a cooling device comprising:
providing a predetermined amount of a phase change composition on a substrate; and
drying the phase change composition to remove substantially all of the water from the composition.

18) The method of claim 17, wherein the predetermined amount of the phase change composition comprises a reversible phase change material and a thermal conductivity additive.

19) The method of claim 17, further comprising applying an adhesive on a side of the substrate opposite the phase change composition.

20) The method of claim 17, further comprising applying a removable backing to the adhesive.
